(19) United States
(12) Patent Application Publication
(10) Pub. No.: US 2021/0059592 A1
(43) Pub. Date: Mar. 4, 2021

(54) DEVICE FOR LOWER EXTREMITY MUSCLE FORCE MEASUREMENT IN ISOKINETIC MOVEMENT CONDITIONS AND PROCEDURE FOR THE MEASUREMENT CARRIED OUT ON THE DEVICE

(71) Applicant: UNIVERZITA KARLOVA, PRAGUE (CZ)

(72) Inventors: FRANTISEK ZAHALKA, PRAGUE (CZ); TOMAS MALY, PRAGUE (CZ); PAVEL VODICKA, PRAGUE (CZ)

(21) Appl. No.: 16/958,678

(22) PCT Filed: Dec. 6, 2018
(86) PCT No.: PCT/CZ2018/050060

§ 371 Date: Jun. 27, 2020

(30) Foreign Application Priority Data
Dec. 29, 2017 (CZ) PV 2017-856

(51) Int. Cl. A61B 5/22; A63B 71/06; A63B 21/002; A63B 24/00; A61B 5/00; A63B 23/02; G01B 11/26

(52) U.S. Cl. CPC A61B 5/224; A63B 71/0622; A63B 21/002; A63B 24/0062; A63B 2208/0214; A63B 23/0211; G01B 11/26; A63B 2220/24; A63B 2220/51; A61B 5/4884

(57) ABSTRACT

The device for lower extremity muscle force measurement in isokinetic movement conditions consists of a supporting surface (3) for knees, angular position sensor (4) equipped with an arm (5), stand with dynamometers (2) and a control unit equipped with an imaging device and containing firmware to measure, record and evaluate data from the angular position sensor (4) and dynamometers (2) connected to it. Procedure for the measurement carried out on the device is defined by the forward angular motion of the measured person kneeling on the supporting surface (3), thus producing pressure on the arm (5) that is pushed in the direction and speed of the measured person's motion, wherein the control unit (6) evaluates the change in angular position of the arm (5) and indicates the current and required speed of change in angular position on the imaging device to maintain the isokinetic movement.

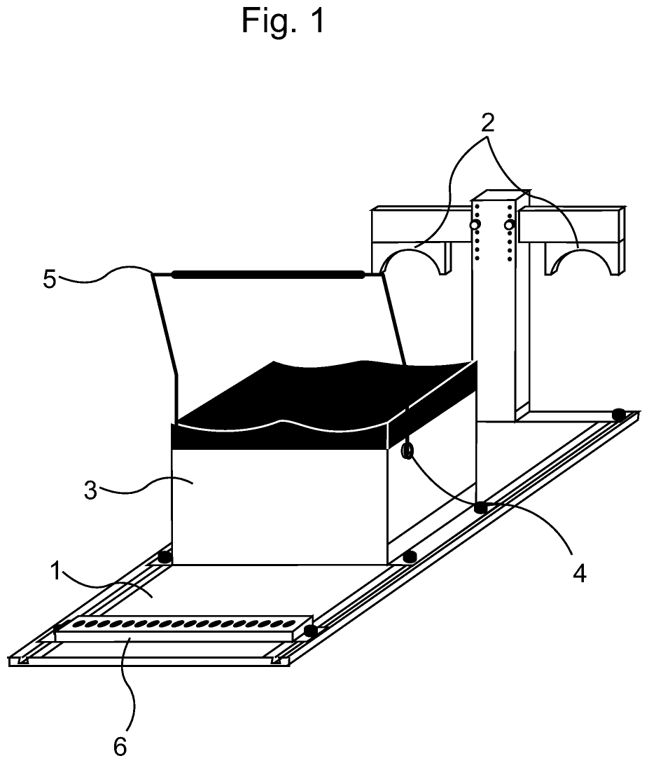

### FIELD OF THE INVENTION

The invention falls within the area of health and physical training technologies, namely devices and methods for kinetic force measurement and muscle training for sports or physiotherapy purposes.

### BACKGROUND OF THE INVENTION

Very complex and therefore large and expensive devices are currently used to measure the kinetic force of lower extremity muscles. Commonly used devices include the device described in WO9111221, where measurement is carried out on a sitting measured person, and therefore has to be provided with a large seat that greatly increases the device weight, thus reducing its mobility. The measured person makes isokinetic movement using the said device for concentric exercise with lower extremity peroneum flexing. So the device has to be provided with electric motors or a hydraulic or pneumatic system, for example, counteracting the force exercised by measured person's muscles. To train lower extremity muscles, devices similar to the above device may be used, or also the work of a physical therapist who carries out the isokinetic exercise, instructing or physically working with the patient. This method of training does not make it possible to measure or evaluate lower extremity muscle force data beyond physical therapist's perception, and therefore cannot be used for measurement due to inaccuracy.

### SUMMARY OF THE INVENTION

The subject of this invention is a device used in particular for lower extremity muscle force measurement or training in isokinetic movement conditions and the procedure for lower extremity muscle force measurement or training using such device. The isokinetic mode ensures muscle motion close to their apex throughout the range of motion in the joint, and enables full contraction at each point of the range of motion. It is essential for the training purpose that the isokinetic movement means maximum active muscle load throughout the range of motion, and enables a significantly greater muscle work compared to ordinary power training as the muscle force is not constant during the motion.

The invention overcomes the drawbacks of the prior art in that it meets the needs relating to kinetic force measurement accuracy in specific muscles in isokinetic movement; thanks to a simpler design, its manufacture and use require considerably lower cost of production and use, and provides better mobility thanks to its smaller size and lower weight. In particular, the invention differs from the prior art in the different position of the measured person's body, who is not sitting with fixed position of the torso and the thigh part of the lower limb, making movements only with the calf part of the lower limb as usual, but, on the contrary, the person has a fixed calf part of the lower limb and carries out the exercise by movements of the torso and the thigh part of the lower limb, where the muscles exert concentric pressure counteracting the pull of gravity.

During measurement or training, lower limb muscles are involved, namely the quadriceps femoris muscle, sartorius muscle, biceps femoris muscle, semitendinosus muscle, semimembranosus muscle and the popliteus muscle, tibialis muscle, extensor digitorum longus muscle, extensor hallucis longus muscle, triceps surae muscle, fibularis longus muscle and the fibularis brevis muscle.

The device consists of a mechanical unit measuring muscle force and a control unit determining the pre-defined isokinetic movement. The mechanical unit consists of a supporting surface for measured person's knees provided with a sensor of measured person's body angular position and an arm connected to the angular position sensor and at least one electric dynamometer to measure the compressive force of muscles. The control unit consists of a computer-controlled system having firmware for evaluating data received from the electric dynamometer and the angular position sensor and the imaging device used to provide visual instructions to the measured person so that the said person maintains isokinetic movements, or alternatively or simultaneously also an audio device for sound instructions. The imaging device may be positioned perpendicular to or in the axis of measured person's movement for better sensomotoric guiding of the measured person. The control unit is also equipped with an interface for data transfer to the computer for their subsequent display and evaluation. Instead of the arm, the angular position sensor may be equipped with an optical sensing device to trace changes in measured person's position. The dynamometers, angular position sensor and the control unit may be powered from an external power supply, or the device may also have its own battery.

If one dynamometer is used, the device enables to measure kinetic muscle force in both lower extremities simultaneously, and if two dynamometers are used, it enables to measure kinetic muscle force in both lower extremities simultaneously or each lower extremity separately.

The described device is used by the measured person being in the kneeling position with vertical position of the torso and the thigh parts of the lower extremities, and the calf parts of the lower extremities perpendicular to them. The calf parts of the lower extremities is thus fixed by the knees on the supporting surface and ankles put up against the dynamometers located above them. The measured person touches the arm of the angular position sensor by the upper thigh area. The measurement or the exercise starts by starting the forward angular motion of the measured person opening the angle between the torso and the thigh parts of the lower extremities and the calf parts of the lower extremities. The torso and the thigh parts of the lower extremities are affected by gravity acting in the forward angular motion direction of this part of the body and is counteracted by the force of lower extremity muscles that is measured in this motion by the dynamometers with the measured person's ankles put up against them. In forward angular motion of the measured person, this motion is copied by the arm of the angular position sensor, which is pushed by the person's motion in the direction and speed of the measured person's motion. The control unit evaluates the progress of measured person's angular motion based on values from the angular position sensor regarding the speed of angular position change, and uses these data to indicate the speed of angular position change and at the same time also the required speed on the imaging device in order to maintain the isokinetic mode. As the motion is carried out, the control unit continuously monitors whether the isokinetic movement mode is maintained, and if the pre-set deviation of actual values from the reference values is exceeded, the control unit evaluates and records the correlation of dynamometer and angular position sensor values. The described procedure for the measurement carried out on the device therefore permits to measure and evaluate the movement process either in terms of the isokinetic movement mode and in terms of achieved individual limits given the physique of the measured person.

### Example of the Embodiment

A preferred embodiment of the device consists of a horizontal surface 1 provided with guide rails in which the following items are inserted to allow the setting of their spacing as separate modules:
1) Stand with two electric dynamometers 2 allowing their vertical setting;
2) Supporting surface 3 to which the angular position sensor 4 is gripped and to which the arm 5 is connected;
3) Control unit 6 with the firmware to evaluate the data from the dynamometers 2 and the angular position sensor 4, equipped with an imaging device.

Procedure for the measurement carried out on the device is implemented in the following steps:
1) The measured person is in the kneeling position on the supporting surface 3 with their torso and the thigh parts of the lower extremities in vertical position and the calf parts of the lower extremities is perpendicular to them, and the ankles are put up against the dynamometers 2 and the upper parts of thighs touch the arm 5 of the angular position sensor 4;
2) The forward motion of the measured person's torso and the thigh parts of the lower extremities produces pressure on the arm 5 of the angular position sensor 4, and the arm 5 thus copies the angular motion of the measured person;
3) The control unit 6 evaluates the change of angular position of the arm 5, and the imaging device indicates the required speed of angular position change so that the isokinetic movement mode is maintained.

### EXPLANATION OF DRAWINGS

The drawing shows a three-quarter view of the device in the configuration as per the preferred embodiment.

### INDUSTRIAL APPLICATION

The device to measure kinetic force of lower extremity muscles can be used primarily for diagnostic or training purposes, for athletes or convalescents.

1. A device for lower extremity muscle force measurement in isokinetic movement conditions, characterized in that it contains a supporting surface (3) for the knees, angular position sensor (4) equipped with a moveable arm (5), at least one dynamometer (2) and a control unit (6) equipped with an imaging device used to provide visual instructions to the measured person placed directly in front of the measured person for visual guidance of the measured person to self-maintain isokinetic movement.

2. The device according to claim 1, characterized in that the dynamometers (2) are placed for vertical adjustment of the dynamometer (2) position.

3. The device according to claim 2, characterized in that the supporting surface (3), the stand with dynamometers (2) and the control unit (6) can be adjusted against each other in the horizontal axis.

4. A method for lower extremity muscle force measurement in isokinetic movement conditions, comprising the steps of:
providing a support surface;
providing an angular position sensor (4) equipped with a moveable arm (5):
providing at least one dynamometer (2); and
providing a control unit (6) equipped with an imaging device;
wherein the measured person is in the kneeling position on the supporting surface (3) with their torso and thigh parts of the lower extremities in a vertical position and the calf parts of the lower extremities are perpendicular to them, and the ankles are put up against the dynamometers (2) and the upper parts of thighs touch the arm (5) of the angular position sensor (4); and
then the forward angular motion of the measured person's torso and thigh parts of the lower extremities produces pressure on the arm (5) of the angular position sensor (4), that is pushed in the direction and speed of the measured person's motion,
wherein the control unit (6) evaluates the change in angular position of the arm (5) of the angular position sensor (4) and provides visually by the imaging device directly to the measured person the information about the current and required speed of change in angular position on the imaging device for the measured person to self-maintain the isokinetic movement.
